(19) United States
(12) Patent Application Publication
(10) Pub. No.: US 2023/0340195 A1
(43) Pub. Date: Oct. 26, 2023

(54) POLYCARBONATE AND METHOD FOR PREPARING SAME

(71) Applicant: LG CHEM, LTD., Seoul (KR)

(72) Inventors: Byunghee Hwang, Daejeon (KR); Bethy Kim, Daejeon (KR); Seoyoung Im, Daejeon (KR); Hoyong Lee, Daejeon (KR); Cheol Jun Song, Daejeon (KR)

(21) Appl. No.: 18/027,866

(22) PCT Filed: Oct. 15, 2021
(86) PCT No.: PCT/KR2021/014344

§ 371 Date: Mar. 22, 2023

(30) Foreign Application Priority Data
Oct. 16, 2020 (KR) 10-2020-0134329

(51) Int. Cl. C08G 64/30; C07D 413/12; C07D 413/14

(52) U.S. Cl. CPC C08G 64/305; C07D 413/12; C07D 413/14

(57) ABSTRACT

The present disclosure relates to polycarbonate a diol compound represented by Chemical Formula 1; at least one compound of compounds represented by Chemical Formulae 2 and 3; and a carbonate precursor-derived repeating unit, and a method for preparing the same.

The present application is a National Phase entry pursuant to 35 U.S.C. § 371 of International Application No. PCT/KR2021/014344 filed on Oct. 15, 2021, and claims priority to and the benefits of Korean Patent Application No. 10-2020-0134329, filed with the Korean Intellectual Property Office on Oct. 16, 2020, the entire contents of which are incorporated herein by reference.

### FIELD

The present disclosure relates to polycarbonate and a method for preparing the same. More specifically, the present disclosure relates to polycarbonate having a novel structure with enhanced flame retardancy, heat resistance, transparency, surface hardness and the like while having excellent mechanical properties.

### BACKGROUND

A polycarbonate resin is a polymer material used in various fields such as exterior materials of electrical and electronic products, automotive parts, construction materials and optical parts due to its properties of excellent impact strength, dimensional stability, heat resistance, transparency and the like.

As the field of application of such a polycarbonate resin has recently expanded into being used in glass and lenses, development of polycarbonate having a novel structure with enhanced weather resistance, refractive index and the like, while maintaining unique properties of a polycarbonate resin itself has been required.

Accordingly, studies to obtain target properties by copolymerizing two or more types of aromatic diol compounds having different structures to introduce units having different structures to the main chain of polycarbonate have been attempted. However, most technologies have limitations such that production costs are high, transparency decreases when chemical resistance, impact strength or the like increases, or chemical resistance, impact strength or the like decreases when transparency is enhanced.

Therefore, research and development on polycarbonate having a novel structure with excellent flame retardancy, heat resistance, transparency, hardness and impact resistance, while having excellent mechanical properties such as surface hardness is still required.

### PATENT DOCUMENTS

(Patent Document 1) International Patent Application Laid-Open Publication No. 99/028387

### SUMMARY

The present disclosure relates to polycarbonate having excellent flame retardancy, heat resistance, hardness and impact resistance while having excellent mechanical properties, and a method for preparing the same.

However, problems that the present disclosure is to resolve are not limited to the problems mentioned above, and other problems not mentioned herein will be clearly understood by those skilled in the art from the following descriptions.

One embodiment of the present disclosure provides polycarbonate including a diol compound represented by the following Chemical Formula 1; at least one compound of compounds represented by the following Chemical Formulae 2 and 3; and a carbonate precursor-derived repeating unit.

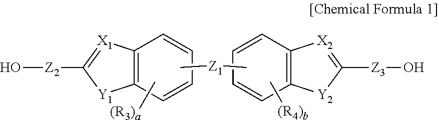

In Chemical Formula 1,
Z₁ is CR₁R₂, O, S, S—S, C═O, C═S, S—O, SO₂, (CH₂)ₙ-L₁-(CH₂)ₘ or O—(C═O),
R₁ and R₂ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic or aromatic ring unsubstituted or substituted with halogen, substituted or unsubstituted alkyl, substituted or unsubstituted aryl or substituted or unsubstituted heteroaryl,
L₁ is O or S,
n and m are each independently an integer of 1 to 3,
Z₂ and Z₃ are each independently a single bond, substituted or unsubstituted alkylene, substituted or unsubstituted cycloalkylene, substituted or unsubstituted arylene or substituted or unsubstituted heteroarylene, or a combination thereof,
X₁ and X₂ are each independently CR₁₀₀ or N,
Y₁ and Y₂ are each independently CR₁₀₁R₁₀₂, O, or S,
R₁₀₀, R₁₀₁, R₁₀₂, R₃ and R₄ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN, COOH or NO₂,
a and b are each independently an integer of 0 to 3, and
R^{a} to R^{f} are each independently hydrogen, substituted or unsubstituted alkyl, substituted or unsubstituted cycloalkyl, substituted or unsubstituted aryl or substituted or unsubstituted heteroaryl,

HO-A₁-OH [Chemical Formula 2]
in Chemical Formula 2,
A₁ is substituted or unsubstituted alkylene, substituted or unsubstituted cycloalkylene or isosorbide,

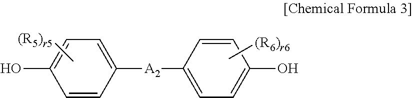

in Chemical Formula 3,
A₂ is substituted or unsubstituted alkylene, substituted or unsubstituted cycloalkylene, substituted or unsubstituted arylene, substituted or unsubstituted heteroarylene, O, S, S—O, SO₂ or C═O,
R⁵ and R⁶ are each independently hydrogen, substituted or unsubstituted alkyl, substituted or unsubstituted cycloalkyl, substituted or unsubstituted aryl, substituted or unsubstituted heteroaryl, alkoxy or halogen, and
r₅ and r₆ are each independently an integer of 0 to 4.

Another embodiment of the present disclosure provides a method for preparing polycarbonate, the method including polymerizing a composition including the diol compound represented by Chemical Formula 1; at least one compound of the compounds represented by Chemical Formulae 2 and 3; and a carbonate precursor.

Another embodiment of the present disclosure provides a molded article including the polycarbonate.

Polycarbonate according to the present disclosure has advantages of having excellent flame retardancy, heat resistance, hardness and impact resistance while having excellent mechanical properties.

Effects of the present disclosure are not limited the above-described effects, and effects not mentioned herein will be clearly understood by those skilled in the art from the specification and accompanying drawings of the present application.

### BRIEF DESCRIPTION OF THE DRAWINGS

FIG. 1 is a ¹H-NMR graph of a diol compound represented by Compound 1 prepared in Example 1.

FIG. 2 is a ¹H-NMR graph of a diol compound represented by Compound 2 prepared in Example 2.

FIG. 3 is a ¹H-NMR graph of a diol compound represented by Compound 3 prepared in Example 3.

### DETAILED DESCRIPTION

Hereinafter, the present disclosure will be described in more detail in order to illuminate the present disclosure.

Polycarbonate and a method for preparing the same according to the present disclosure will be described hereinafter, however, unless defined otherwise, technical terms and scientific terms used herein have meanings commonly understandable to those skilled in the art relating to the present disclosure, and in the following descriptions, descriptions on known functions and constitutions that may unnecessarily obscure the gist of the present disclosure will not be included.

Terms or words used in the descriptions and the claims of the present disclosure are not to be interpreted limitedly to common or dictionary meanings, and shall be interpreted as meanings and concepts corresponding to technological ideas of the present disclosure based on a principle in which inventors may suitably define the concepts of terms in order to describe the invention in the best possible way.

Throughout the specification of the present application, a description of a certain part “including” certain constituents means capable of further including other constituents, and does not exclude other constituents unless particularly stated on the contrary.

Throughout the specification of the present application, a description of a certain member being placed “on” another member includes not only a case of the certain member being in contact with the another member but a case of still another member being present between the two members.

Throughout the specification of the present application, “parts by weight” may mean a weight ratio between each component.

Throughout the specification of the present application, “molar ratio” refers to a ratio of a molar equivalent of X with respect to a molar equivalent of Y, and X and Y herein may be, for example, each component in a reaction mixture.

Throughout the specification of the present application, “one or more” means, for example, “1, 2, 3, 4 or 5, particularly 1, 2, 3 or 4, more particularly 1, 2 or 3, and even more particularly 1 or 2”.

In the present specification, “*” means a bond linked to other substituents. In one example, “*” means a part linked to other substituents to form a spiro ring.

In the present specification,

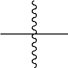

means a bond linked to other substituents or linking sites.

Throughout the specification of the present application, a weight average molecular weight (Mw), a number average molecular weight (Mn) and a Z average molecular weight (Mz+1) are numbers converted with respect to standard polystyrene measured using gel permeation chromatography (GPC, manufactured by Waters). However, the weight average molecular weight (Mw), the number average molecular weight (Mn) and the Z average molecular weight (Mz+1) are not limited thereto, and may be measured using other methods known in the art.

In the present specification, “*” means a bond linked to other substituents.

In the present specification, a term “single bond” means a direct bond.

In the present specification, a term “derived repeating unit” means, when polymerizing a polymer, a repeating unit formed by introduced monomers in the polymer participating in the polymerization reaction.

Throughout the specification of the present application, “substituted or unsubstituted” means being substituted with one or more substituents selected from the group consisting of deuterium; a halogen group; a cyano group; a nitrile group; a nitro group; a hydroxyl group; an alkoxy group; a cycloalkoxy group; an aryloxy group; a heterocyclyloxy group; a haloalkyl group; an alkyl group; a cycloalkyl group; an alkenyl group; an alkynyl group; an aryl group; and a heteroaryl group including one or more of N, O and S atoms or being unsubstituted, or being substituted with a substituent linking two or more substituents among the substituents illustrated above or being unsubstituted.

Throughout the specification of the present application, the “substituent linking two or more substituents” may be a biphenyl group. In other words, a biphenyl group may be an aryl group, or interpreted as a substituent linking two phenyl groups.

In the present specification, the term “deuterium” refers to a stable isotope of hydrogen having a mass approximately twice that of a most common isotope, that is, a mass of approximately 2 atomic mass units.

Throughout the specification of the present application, the “halogen group” refers to a fluoro (F), a chloro (Cl), a bromo (Br) or an iodo (I) atom.

In the present specification, the term “cyano group” or “nitrile group” means a —C≡N group.

Throughout the specification of the present application, an “isocyanate group” means a —N≡C═O group.

Throughout the specification of the present application, the “nitro group” refers to a —NO₂ group.

Throughout the specification of the present application, the “hydroxyl group” refers to an —OH group.

Throughout the specification of the present application, the “alkoxy group”, the “cycloalkoxy group”, the “aryloxy group” and the “heterocyclyloxy group” refer to any one of the alkyl, the cycloalkyl, the aryl or the heterocyclyl attached to the rest of the molecule through an oxygen atom (—O—). Herein, the alkyl, the cycloalkyl, the aryl or the heterocyclyl is substituted or unsubstituted.

Throughout the specification of the present application, an “alkylthioxy group” and an “arylthioxy group” refer to any one of the alkyl or the aryl attached to the rest of the molecule through a sulfur atom (—S—).

Throughout the specification of the present application, an “aliphatic ring” means a saturated or unsaturated non-aromatic monocyclic, bicyclic or tricyclic hydrocarbon site of cyclic carbon having 5 to 14, 5 to 10, or 5 to 6 carbon atoms, and, although not limited thereto, examples thereof may include a cycloalkane ring such as a cyclopentane ring or a cyclohexane ring, a cycloalkene ring such as a cyclopentene ring, a cyclohexene ring or a cyclooctene ring, and the like. The aliphatic ring is an aliphatic hydrocarbon ring or an aliphatic heteroring.

Throughout the specification of the present application, an “aromatic ring” is an aryl ring or a heteroaryl ring, and descriptions on the aryl and the heteroaryl are the same as descriptions to provide later.

Throughout the specification of the present application, “isosorbide” is a 100% natural biomaterial made from corn, and may include isosorbide isomers with no particular limit in the stereochemistry.

Throughout the specification of the present application, the “alkyl group” means linear or branched saturated hydrocarbon. Specifically, the number of carbon atoms of the alkyl group is not particularly limited, but is preferably from 1 to 40. According to one embodiment, the number of carbon atoms of the alkyl group is from 1 to 20. According to another embodiment, the number of carbon atoms of the alkyl group is from 1 to 10. According to another embodiment, the number of carbon atoms of the alkyl group is from 1 to 6. Specific examples of the alkyl group may include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl and the like, but are not limited thereto.

Throughout the specification of the present application, the “haloalkyl group” means the alkyl group being substituted with at least one halogen group.

Throughout the specification of the present application, the “cycloalkyl group” refers to a completely saturated and partially unsaturated hydrocarbon ring of carbon atoms. Specifically, the cycloalkyl group is not particularly limited, but preferably has 3 to 60 carbon atoms, and according to one embodiment, the number of carbon atoms of the cycloalkyl group is from 3 to 30. According to another embodiment, the number of carbon atoms of the cycloalkyl group is from 3 to 20. According to another embodiment, the number of carbon atoms of the cycloalkyl group is from 3 to 6. Specific examples thereof may include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl and the like, but are not limited thereto.

Throughout the specification of the present application, the “alkenyl group” refers to linear or branched unsaturated hydrocarbon including one or more double bonds. Specifically, the alkenyl group may be linear or branched, and although not particularly limited thereto, the number of carbon atoms is preferably from 2 to 40. According to one embodiment, the number of carbon atoms of the alkenyl group is from 2 to 20. According to another embodiment, the number of carbon atoms of the alkenyl group is from 2 to 10. According to another embodiment, the number of carbon atoms of the alkenyl group is from 2 to 6. Specific examples thereof may include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group and the like, but are not limited thereto.

Throughout the specification of the present application, the “alkynyl group” means a linear or branched unsaturated hydrocarbon radical including one or more triple bonds. Specifically, the alkynyl group may be linear or branched, and although not particularly limited thereto, the number of carbon atoms is preferably from 2 to 40. According to one embodiment, the number of carbon atoms of the alkynyl group is from 2 to 20. According to another embodiment, the number of carbon atoms of the alkynyl group is from 2 to 10. According to another embodiment, the number of carbon atoms of the alkynyl group is from 2 to 6. Specific examples thereof may include short-chain hydrocarbon radicals selected from among ethynyl, prop-1-yn-1-yl, prop-2-yn-1-yl, but-1-yn-1-yl, but-1-yn-3-yl, but-3-yn-1-yl and the like, but are not limited thereto.

Throughout the specification of the present application, the “alkylene group” means linear or branched divalent aliphatic saturated hydrocarbon. Specifically, the alkylene group may mean divalent aliphatic saturated hydrocarbon such as methylene, ethylene, propylene and butylene, but is not limited thereto.

Throughout the specification of the present application, the “aryl group” means, as an organic radical derived from aromatic hydrocarbon by removing one hydrogen, a monocyclic or polycyclic aromatic hydrocarbon radical. Specifically, the aryl group is not particularly limited, but preferably has 6 to 60 carbon atoms, and may be a monocyclic aryl group or a polycyclic aryl group. According to one embodiment, the number of carbon atoms of the aryl group is from 6 to 30. According to one embodiment, the number of carbon atoms of the aryl group is from 6 to 20. When the aryl group is a monocyclic aryl group, examples thereof may include a phenyl group, a biphenyl group, a terphenyl group and the like, but are not limited thereto. When the aryl group is a polycyclic aryl group, examples thereof may include a naphthyl group, an anthracenyl group, a phenanthryl group, a pyrenyl group, a perylenyl group, a chrysenyl group, a fluorenyl group and the like, but are not limited thereto.

Throughout the specification of the present application, the “fluorenyl group” means a 9-fluorenyl radical.

Specifically, the fluorenyl group may be substituted, and two substituents may bond to each other to form a spiro structure. When the fluorenyl group is substituted,

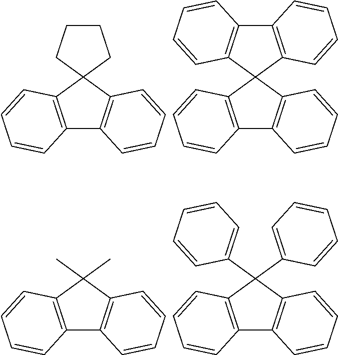

and the like may be included. However, the structure is not limited thereto.

Throughout the specification of the present application, the “heteroaryl group” means, as an organic radical derived from aromatic hydrocarbon by removing one hydrogen, heteroaryl including one or more heteroatoms selected from among B, N, O, S, P(═O), Si and P. Specifically, the number of carbon atoms of the heteroaryl group is not particularly limited, but is preferably from 3 to 60. Examples of the heteroaryl group may include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, a triazole group, an acridly group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzimidazole group, a benzothiazole group, a benzocarbazole group, a benzothiophene group, a dibenzothiophene group, a benzofuranyl group, a phenanthroline group, a thiazolyl group, an isoxazolyl group, an oxadiazolyl group, a thiadiazolyl group, a benzothiazolyl group, a phenothiazinyl group, a dibenzofuranyl group and the like, but are not limited thereto.

In the present specification, the descriptions on the aryl group provided above may be applied to the arylene except that the arylene is a divalent group.

In the present specification, the descriptions on the heteroaryl group provided above may be applied to the heteroarylene except that the heteroarylene is a divalent group.

### Polycarbonate

A repeating unit derived from a diol compound represented by Chemical Formula 1 enhances hardness of polycarbonate, a repeating unit derived from a compound represented by Chemical Formula 2 enhances transparency polycarbonate, and a repeating unit derived from a compound represented by Chemical Formula 3 enhances heat resistance of polycarbonate. Accordingly, polycarbonate having target properties may be prepared by, while including at least one of the compound represented by Chemical Formula 2 and the compound represented by Chemical Formula 3-derived repeating units with the diol compound represented by Chemical Formula 1-derived repeating unit, properly adjusting a molar ratio of the compounds.

One embodiment of the present disclosure provides polycarbonate including a diol compound represented by the following Chemical Formula 1; at least one compound of compounds represented by the following Chemical Formulae 2 and 3; and a carbonate precursor-derived repeating unit.

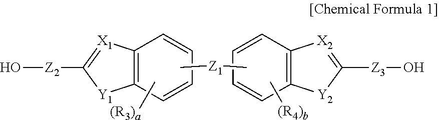

In Chemical Formula 1,
Z₁ is CR₁R₂, O, S, S—S, C═O, C═S, S—O, SO₂, (CH₂)ₙ-L₁-(CH₂)ₘ or O—(C═O),
R₁ and R₂ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic or aromatic ring unsubstituted or substituted with halogen, substituted or unsubstituted alkyl, substituted or unsubstituted aryl or substituted or unsubstituted heteroaryl,
L₁ is O or S,
n and m are each independently an integer of 1 to 3,
Z₂ and Z₃ are each independently a single bond, substituted or unsubstituted alkylene, substituted or unsubstituted cycloalkylene, substituted or unsubstituted arylene or substituted or unsubstituted heteroarylene, or a combination thereof,
X₁ and X₂ are each independently CR₁₀₀ or N,
Y₁ and Y₂ are each independently CR₁₀₁R₁₀₂, O or S,
R₁₀₀, R₁₀₁, R₁₀₂, R₃ and R₄ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂,
a and b are each independently an integer of 0 to 3, and
R^{a} to R^{f} are each independently hydrogen, substituted or unsubstituted alkyl, substituted or unsubstituted cycloalkyl, substituted or unsubstituted aryl or substituted or unsubstituted heteroaryl,

HO-A₁-OH [Chemical Formula 2]
in Chemical Formula 2,
A₁ is substituted or unsubstituted alkylene, substituted or unsubstituted cycloalkylene or isosorbide,

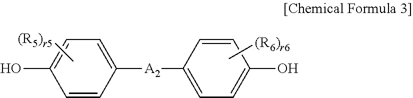

in Chemical Formula 3,
A₂ is substituted or unsubstituted alkylene, substituted or unsubstituted cycloalkylene, substituted or unsubstituted arylene, substituted or unsubstituted heteroarylene, O, S, S—O, SO₂ or C═O,
R₅ and R₆ are each independently hydrogen, substituted or unsubstituted alkyl, substituted or unsubstituted cycloalkyl, substituted or unsubstituted aryl, substituted or unsubstituted heteroaryl, alkoxy or halogen, and
r₅ and r₆ are each independently an integer of 0 to 4.

In the present disclosure, the polycarbonate includes the diol compound represented by Chemical Formula 1; the compound represented by Chemical Formula 3; and a carbonate precursor-derived repeating unit.

In the present disclosure, in Chemical Formula 1, Z₁ is CR₁R₂, O, S, S—S, C═O, C═S, S—O, SO₂, (CH₂)ₙ-L₁-(CH₂)ₘ or O—(C═O),
R₁ and R₂ are each independently hydrogen, substituted or unsubstituted alkyl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic ring of

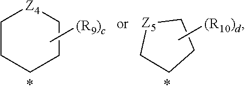

and * means a part linked to Chemical Formula 1,
L₁ is O or S,
n and m are each independently an integer of 1 to 3,
Z₄ and Z₅ are each independently CR₁₀₃R₁₀₄, NR₁₀₅, O or S,
R₁₀₃ to R₁₀₅, R₉ and R₁₀ are each independently hydrogen, halogen, substituted or unsubstituted alkyl, substituted or unsubstituted aryl or substituted or unsubstituted heteroaryl, and
c is an integer of 0 to 8, and d is an integer of 0 to 6.

In the present disclosure, R₁ and R₂ are linked to form an aliphatic ring of

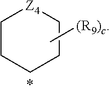

In the present disclosure, Z₄ is S.

In the present disclosure, R₉ is hydrogen.

In the present disclosure, Z₂ and Z₃ are each independently

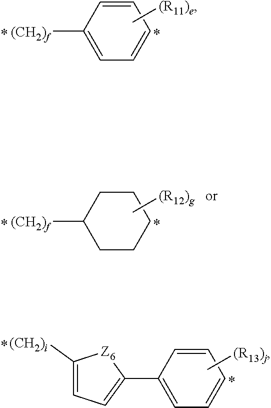

* means a part linked to Chemical Formula 1,
R₁₁ to R₁₃ are each independently hydrogen, alkoxy, substituted or unsubstituted alkyl or OH,
Z₆ is NRn, O or S,
Rn is hydrogen or a substituted or unsubstituted alkyl group,
e and j are each independently an integer of 0 to 4,
g is an integer of 0 to 10, and
f, h and i are each independently an integer of 0 to 10.

In the present disclosure, when X₁ and X₂ are N, Y₁ and Y₂ are each independently O or S, and when X₁ and X₂ are CR₁₀₀, Y₁ and Y₂ are each independently CR₁₀₁R₁₀₂ or O.

In the present disclosure, in Chemical Formula 1, R₃ and R₄ are each independently hydrogen, substituted or unsubstituted alkyl, halogen, CN or NO₂.

In the present disclosure, Z₁ is CR₁R₂, C═O or SO₂.

In the present disclosure, Z₁ is CR₁R₂.

In the present disclosure, Z₁ is C═O.

In the present disclosure, Z₁ is SO₂.

In the present disclosure, R₁ and R₂ are each independently hydrogen, substituted or unsubstituted alkyl, substituted or unsubstituted haloalkyl, OR^{a}, SR^{b}, COOR^{f}, halogen or CN, or R₁ and R₂ are linked to each other to form an aliphatic ring.

In the present disclosure, R₁ and R₂ are each independently hydrogen, substituted or unsubstituted alkyl having 1 to 30 carbon atoms, substituted or unsubstituted haloalkyl having 1 to 30 carbon atoms, OR^{a}, SR^{b}, COOR^{f}, halogen or CN, or R₁ and R₂ are linked to each other to form an aliphatic ring having 1 to 30 carbon atoms.

In the present disclosure, R₁ and R₂ are each independently hydrogen, substituted or unsubstituted alkyl having 1 to 20 carbon atoms, substituted or unsubstituted haloalkyl having 1 to 20 carbon atoms, OR^{a}, SR^{b}, COOR^{f}, halogen or CN, or R₁ and R₂ are linked to each other to form an aliphatic ring having 1 to 20 carbon atoms.

In the present disclosure, R₁ and R₂ are each independently hydrogen, substituted or unsubstituted alkyl having 1 to 10 carbon atoms, substituted or unsubstituted haloalkyl having 1 to 10 carbon atoms, OR^{a}, SR^{b}, COOR^{f}, halogen or CN, or R₁ and R₂ are linked to each other to form an aliphatic ring having 1 to 10 carbon atoms.

In the present disclosure, R₁ and R₂ are each independently hydrogen, a methyl group, a trifluoromethyl group, OR^{a}, SR^{b}, COOR^{f}, halogen or CN, or R₁ and R₂ are linked to each other to form an aliphatic ring of

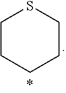

In the present disclosure, R₁ and R₂ are a trifluoromethyl group or a methyl group.

In the present disclosure, R^{a} to R^{f} are each independently hydrogen, substituted or unsubstituted alkyl having 1 to 30 carbon atoms, substituted or unsubstituted cycloalkyl having 3 to 30 carbon atoms, substituted or unsubstituted aryl having 6 to 30 carbon atoms or substituted or unsubstituted heteroaryl having 2 to 30 carbon atoms.

In the present disclosure, R^{a} to R^{f} are each independently hydrogen, substituted or unsubstituted alkyl having 1 to 20 carbon atoms, substituted or unsubstituted cycloalkyl having 3 to 20 carbon atoms, substituted or unsubstituted aryl having 6 to 20 carbon atoms or substituted or unsubstituted heteroaryl having 2 to 20 carbon atoms.

In the present disclosure, R^{a} to R^{f} are each independently hydrogen, substituted or unsubstituted alkyl having 1 to 10 carbon atoms, substituted or unsubstituted cycloalkyl having 3 to 10 carbon atoms, substituted or unsubstituted aryl having 6 to 10 carbon atoms or substituted or unsubstituted heteroaryl having 2 to 10 carbon atoms.

In the present disclosure, R^{a}, R^{b} or R^{f} is each independently hydrogen or substituted or unsubstituted alkyl.

In the present disclosure, R^{a}, R^{b} or R^{f} is each independently hydrogen or substituted or unsubstituted alkyl having 1 to 30 carbon atoms.

In the present disclosure, R^{a}, R^{b} or R^{f} is each independently hydrogen or substituted or unsubstituted alkyl having 1 to 20 carbon atoms.

In the present disclosure, R^{a}, R^{b} or R^{f} is each independently hydrogen or substituted or unsubstituted alkyl having 1 to 10 carbon atoms.

In the present disclosure, R^{a}, R^{b} or R^{f} is each independently hydrogen or a substituted or unsubstituted methyl group.

In the present disclosure, R^{a}, R^{b} or R^{f} is each independently hydrogen or a methyl group.

In the present disclosure, Z₂ and Z₃ are each independently a single bond, substituted or unsubstituted alkylene having 1 to 30 carbon atoms, substituted or unsubstituted cycloalkylene having 3 to 30 carbon atoms, substituted or unsubstituted arylene having 6 to 30 carbon atoms or substituted or unsubstituted heteroarylene having 2 to 30 carbon atoms, or a combination thereof.

In the present disclosure, Z₂ and Z₃ are each independently a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms or substituted or unsubstituted heteroarylene having 2 to 20 carbon atoms, or a combination thereof.

In the present disclosure, Z₂ and Z₃ are each independently a single bond, substituted or unsubstituted alkylene having 1 to 10 carbon atoms, substituted or unsubstituted cycloalkylene having 3 to 10 carbon atoms, substituted or unsubstituted arylene having 6 to 10 carbon atoms or substituted or unsubstituted heteroarylene having 2 to 10 carbon atoms, or a combination thereof.

In the present disclosure, Z₂ and Z₃ are each independently substituted or unsubstituted cyclohexylene, substituted or unsubstituted cyclohexylene-substituted or unsubstituted methylene, substituted or unsubstituted phenylene, substituted or unsubstituted phenylene-substituted or unsubstituted methylene, or substituted or unsubstituted phenylene-substituted or unsubstituted divalent thiophene-substituted or unsubstituted methylene.

In the present disclosure, Z₂ and Z₃ are each independently cyclohexylene; cyclohexylene-methylene; phenylene unsubstituted or substituted with a methyl group, or a methoxy group; phenylene-methylene; or phenylene-divalent thiophene-methylene.

In the present disclosure, X₁ and X₂ are CR₁₀₀, and R₁₀₀ is hydrogen.

In the present disclosure, X₁ and X₂ are N.

In the present disclosure, Y₁ and Y₂ are CR₁₀₁R₁₀₂, and R₁₀₁ and R₁₀₂ are hydrogen.

In the present disclosure, Y₁ and Y₂ are O.

In the present disclosure, Y₁ and Y₂ are S.

In the present disclosure, R₁₀₀, R₁₀₃, R₁₀₂, R₃ and R₄ are each independently hydrogen, substituted or unsubstituted alkyl having 1 to 30 carbon atoms, substituted or unsubstituted cycloalkyl having 3 to 30 carbon atoms, substituted or unsubstituted aryl having 6 to 30 carbon atoms, substituted or unsubstituted heteroaryl having 2 to 30 carbon atoms, substituted or unsubstituted haloalkyl having 1 to 30 carbon atoms, OR^{a}, SR^{b}, NR^{c}R^{d}, COOR^{e}, OCOR^{f}, halogen, CN, COOH or NO₂.

In the present disclosure, R₁₀₀, R₁₀₁, R₁₀₂, R₃ and R₄ are each independently hydrogen, substituted or unsubstituted alkyl having 1 to 20 carbon atoms, substituted or unsubstituted cycloalkyl having 3 to 20 carbon atoms, substituted or unsubstituted aryl having 6 to 20 carbon atoms, substituted or unsubstituted heteroaryl having 2 to 20 carbon atoms, substituted or unsubstituted haloalkyl having 1 to 20 carbon atoms, OR^{a}, SR^{b}, NR^{c}R^{d}, COOR^{e}, OCOR^{f}, halogen, CN, COOH or NO₂.

In the present disclosure, R₁₀₀, R₁₀₁, R₁₀₂, R₃ and R₄ are each independently hydrogen, substituted or unsubstituted alkyl having 1 to 10 carbon atoms, substituted or unsubstituted cycloalkyl having 3 to 10 carbon atoms, substituted or unsubstituted aryl having 6 to 10 carbon atoms, substituted or unsubstituted heteroaryl having 2 to 10 carbon atoms, substituted or unsubstituted haloalkyl having 1 to 10 carbon atoms, OR^{a}, SR^{b}, NR^{c}R^{d}, COOR^{e}, OCOR^{f}, halogen, CN, COOH or NO₂.

In the present disclosure, R₃ and R₄ are each independently hydrogen, halogen, CN or substituted or unsubstituted alkyl.

In the present disclosure, R₃ and R₄ are each independently hydrogen, halogen, CN or substituted or unsubstituted alkyl having 1 to 30 carbon atoms.

In the present disclosure, R₃ and R₄ are each independently hydrogen, halogen, CN or substituted or unsubstituted alkyl having 1 to 20 carbon atoms.

In the present disclosure, R₃ and R₄ are each independently hydrogen, halogen, CN or substituted or unsubstituted alkyl having 1 to 10 carbon atoms.

In the present disclosure, R₃ and R₄ are each independently hydrogen, bromine, CN or a substituted or unsubstituted methyl group.

In the present disclosure, R₃ and R₄ are each independently hydrogen, bromine, CN or a methyl group.

In the present disclosure, R₁₀₀ is hydrogen.

In the present disclosure, R₁₀₁ and R₁₀₂ are hydrogen.

In the present disclosure, A₁ is substituted or unsubstituted alkylene having 1 to 30 carbon atoms, substituted or unsubstituted cycloalkylene having 3 to 30 carbon atoms or isosorbide.

In the present disclosure, A₁ is substituted or unsubstituted alkylene having 1 to 20 carbon atoms, substituted or unsubstituted cycloalkylene having 3 to 20 carbon atoms or isosorbide.

In the present disclosure, A₁ is substituted or unsubstituted alkylene having 1 to 10 carbon atoms, substituted or unsubstituted cycloalkylene having 3 to 10 carbon atoms or isosorbide.

In the present disclosure, A₁ is isosorbide.

In the present disclosure, A₂ is substituted or unsubstituted alkylene having 1 to 30 carbon atoms, substituted or unsubstituted cycloalkylene having 3 to 30 carbon atoms, substituted or unsubstituted arylene having 6 to 30 carbon atoms, substituted or unsubstituted heteroarylene having 2 to 30 carbon atoms, O, S, S—O, SO₂ or C═O.

In the present disclosure, A₂ is substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 2 to 20 carbon atoms, O, S, S—O, SO₂ or C═O.

In the present disclosure, A₂ is substituted or unsubstituted alkylene having 1 to 20 carbon atoms, substituted or unsubstituted cycloalkylene having 3 to 10 carbon atoms, substituted or unsubstituted arylene having 6 to 10 carbon atoms, substituted or unsubstituted heteroarylene having 2 to 10 carbon atoms, O, S, S—O, SO₂ or C═O.

In the present disclosure, A₂ is substituted or unsubstituted methylene.

In the present disclosure, A₂ is methylene substituted with a methyl group.

In the present disclosure, R₅ and R₆ are each independently hydrogen, substituted or unsubstituted alkyl having 1 to 30 carbon atoms, substituted or unsubstituted cycloalkyl having 3 to 30 carbon atoms, substituted or unsubstituted aryl having 6 to 30 carbon atoms, substituted or unsubstituted heteroaryl having 2 to 30 carbon atoms, alkoxy or halogen.

In the present disclosure, R₅ and R₆ are each independently hydrogen, substituted or unsubstituted alkyl having 1 to 20 carbon atoms, substituted or unsubstituted cycloalkyl having 3 to 20 carbon atoms, substituted or unsubstituted aryl having 6 to 20 carbon atoms, substituted or unsubstituted heteroaryl having 2 to 20 carbon atoms, alkoxy or halogen.

In the present disclosure, R₅ and R₆ are each independently hydrogen, substituted or unsubstituted alkyl having 1 to 10 carbon atoms, substituted or unsubstituted cycloalkyl having 3 to 10 carbon atoms, substituted or unsubstituted aryl having 6 to 10 carbon atoms, substituted or unsubstituted heteroaryl having 2 to 10 carbon atoms, alkoxy or halogen.

In the present disclosure, R₅ and R₆ are hydrogen.

In the present disclosure, R₁₁ to R₁₃ are each independently hydrogen, alkoxy, substituted or unsubstituted alkyl having 1 to 30 carbon atoms or OH.

In the present disclosure, R₁₁ to R₁₃ are each independently hydrogen, alkoxy, substituted or unsubstituted alkyl having 1 to 20 carbon atoms or OH.

In the present disclosure, R₁₁ to R₁₃ are each independently hydrogen, alkoxy, substituted or unsubstituted alkyl having 1 to 10 carbon atoms or OH.

In the present disclosure, R₁₁ to R₁₃ are each independently hydrogen, methoxy, substituted or unsubstituted methyl or OH.

In the present disclosure, R₁₁ to R₁₃ are each independently hydrogen, methoxy, methyl or OH.

In the present disclosure, the diol compound represented by Chemical Formula 1 may be as follows, but is not limited thereto.

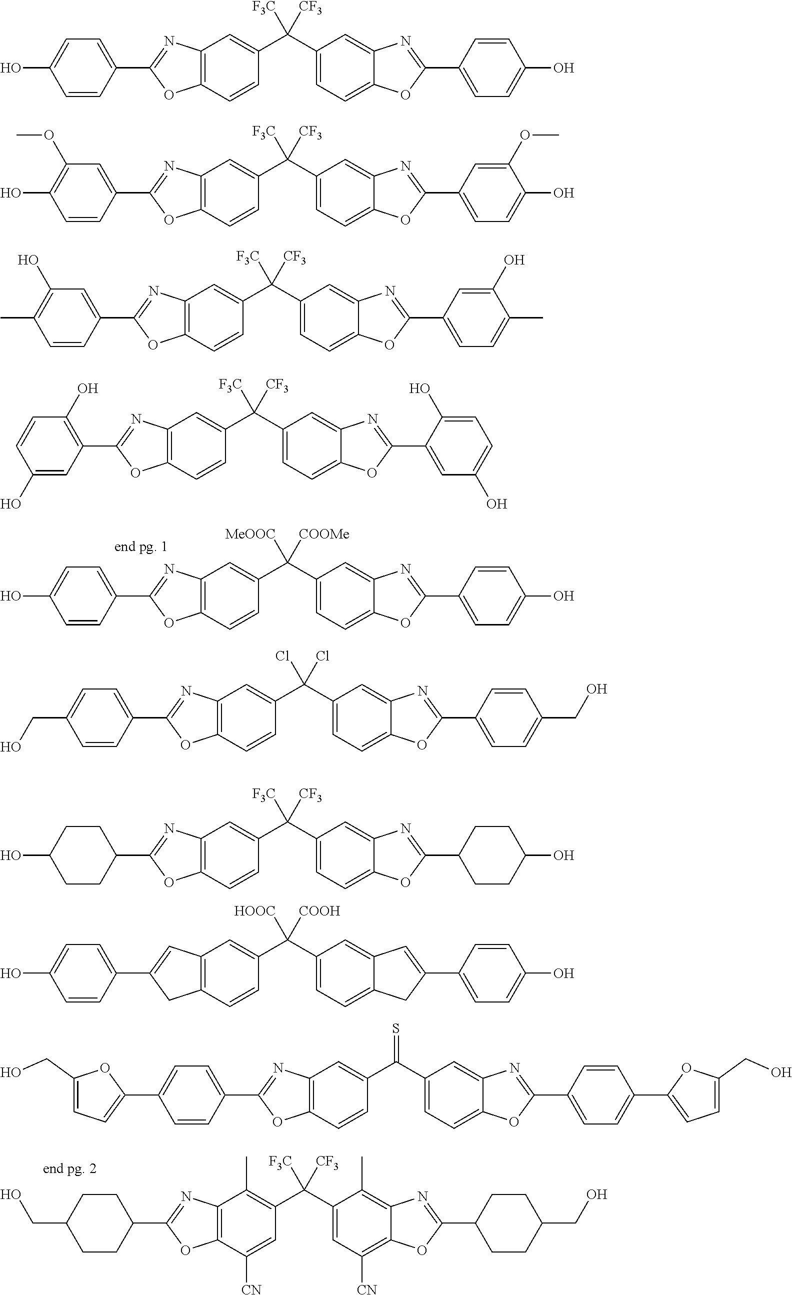

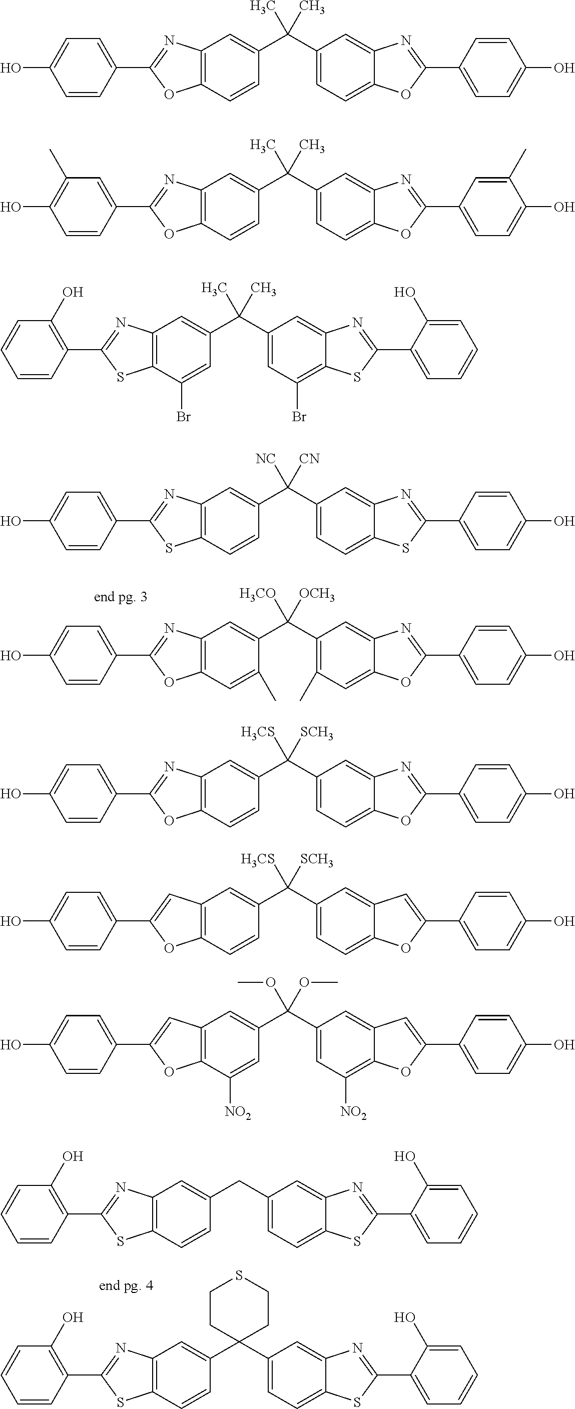

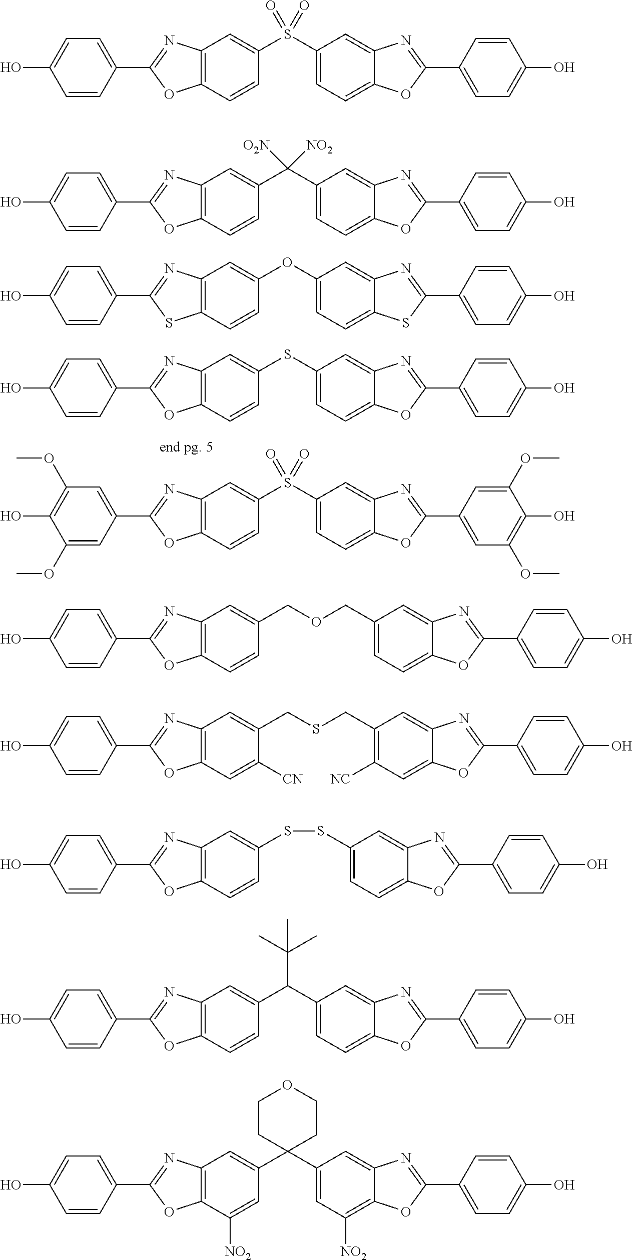

In the present disclosure, the diol compound represented by Chemical Formula 1; at least one compound of the compounds represented by Chemical Formulae 2 and 3; and the carbonate precursor-derived repeating unit include a unit represented by the following Chemical Formula 4.

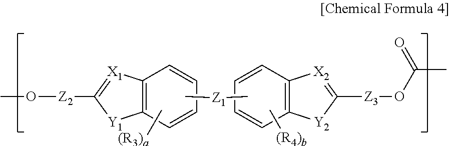

In Chemical Formula 4,
Z₁ to Z₃, R₃, R₄, X₁, X₂, Y₁, Y₂, a and b have the same definitions as in Chemical Formula 1.

In the present disclosure, the diol compound represented by Chemical Formula 1; at least one compound of the compounds represented by Chemical Formulae 2 and 3; and the carbonate precursor-derived repeating unit further includes a repeating unit represented by the following Chemical Formula 5.

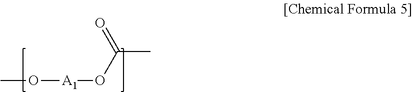

In Chemical Formula 5,
A₁ has the same definition as in Chemical Formula 2.

In the present disclosure, the diol compound represented by Chemical Formula 1; at least one compound of the compounds represented by Chemical Formulae 2 and 3; and the carbonate precursor-derived repeating unit further includes a repeating unit represented by the following Chemical Formula 6.

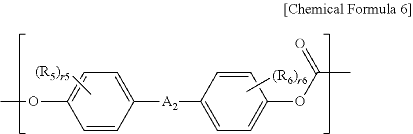

In Chemical Formula 6,
A₂, R₅, R₆, r₅ and r₆ have the same definitions as in Chemical Formula 3.

When the polycarbonate of the present disclosure further includes the compound represented by Chemical Formula 2-derived repeating unit in addition to the diol compound represented by Chemical Formula 1-derived repeating unit, a molar ratio thereof is not particularly limited, and for example, the diol compound represented by Chemical Formula 1-derived repeating unit and the compound represented by Chemical Formula 2-derived repeating unit may have a molar ratio of 99:1 to 1:99. In specific examples, the diol compound represented by Chemical Formula 1-derived repeating unit and the compound represented by Chemical Formula 2-derived repeating unit may have a molar ratio of 50:50 to 3:97, or 30:70 to 5:95, or 15:85 to 10:90.

When the diol compound represented by Chemical Formula 1-derived repeating unit and the compound represented by Chemical Formula 2-derived repeating unit satisfy the molar ratio in the above-described range, hardness of the polycarbonate is superior, transparency is superior, and polycarbonate productivity is superior by maintaining reactivity.

When the polycarbonate of the present disclosure further includes the compound represented by Chemical Formula 3-derived repeating unit in addition to the diol compound represented by Chemical Formula 1-derived repeating unit, a molar ratio thereof is not particularly limited, and for example, the diol compound represented by Chemical Formula 1-derived repeating unit and the compound represented by Chemical Formula 3-derived repeating unit may have a molar ratio of 99:1 to 1:99. In specific examples, the diol compound represented by Chemical Formula 1-derived repeating unit and the compound represented by Chemical Formula 3-derived repeating unit may have a molar ratio of 50:50 to 3:97, or 30:70 to 5:95, or 15:85 to 10:90.

When the diol compound represented by Chemical Formula 1-derived repeating unit and the compound represented by Chemical Formula 3-derived repeating unit satisfy the molar ratio in the above-described range, hardness of the polycarbonate is superior, transparency is superior, and polycarbonate productivity is superior by maintaining reactivity.

When the polycarbonate of the present disclosure further includes the compound represented by Chemical Formula 2-derived repeating unit and the compound represented by Chemical Formula 3-derived repeating unit in addition to the diol compound represented by Chemical Formula 1-derived repeating unit, a molar ratio thereof is not particularly limited, and for example, the diol compound represented by Chemical Formula 1-derived repeating unit, the compound represented by Chemical Formula 2-derived repeating unit and the compound represented by Chemical Formula 3-derived repeating unit may have a molar ratio of 10:10:80 to 80:10:10. In specific examples, the diol compound represented by Chemical Formula 1-derived repeating unit, the compound represented by Chemical Formula 2-derived repeating unit and the compound represented by Chemical Formula 3-derived repeating unit may have a molar ratio of 10:10:80 to 80:10:10, or 10:80:10.

When the diol compound represented by Chemical Formula 1-derived repeating unit, the compound represented by Chemical Formula 2-derived repeating unit and the compound represented by Chemical Formula 3-derived repeating unit satisfy the molar ratio in the above-described range, hardness of the polycarbonate is superior, transparency is superior, and polycarbonate productivity is superior by maintaining reactivity.

In the present disclosure, a weight average molecular weight (Mw) of the polycarbonate may be properly adjusted depending on purposes and applications, and the weight average molecular weight (Mw) measured by GPC using a PC standard may be from 1,000 g/mol to 100,000 g/mol, preferably from 10,000 g/mol to 100,000 g/mol, and more preferably from 10,000 g/mol to 50,000 g/mol or 40,000 g/mol to 48,000 g/mol. In one example, mechanical properties of the polycarbonate may not be sufficient when the weight average molecular weight (Mw) is less than 1,000 g/mol, and when the weight average molecular weight (Mw) is greater than 100,000 g/mol, productivity of the polycarbonate may be reduced.

In the present disclosure, a melt index of the polycarbonate measured in accordance with the ASTM D1238 (condition of 300° C., 1.2 kg) may be properly adjusted depending on purposes and applications, and the melt index may be 1 g/10 min or greater, 3 g/10 min or greater or 8 g/10 min or greater, and 100 g/10 min or less, 30 g/10 min or less or 15 g/10 min or less.

In the present disclosure, Izod room temperature impact strength of the polycarbonate measured at 23° C. in accordance with the ASTM D256 (⅛ inch, Notched Izod) is 220 Kgf/m² or greater. The Izod room temperature impact strength may be 230 Kgf/m² or greater, 240 Kgf/m² or greater, 245 Kgf/m² or greater or 250 Kgf/m² or greater, and 1,000 Kgf/m² or less, 500 Kgf/m² or less, 400 Kgf/m² or less or 310 Kgf/m² or less.

In the present disclosure, a glass transition temperature (Tg) of the polycarbonate satisfies 150° C. or higher, 153° C. or higher, 154° C. or higher or 155° C. higher, and 190° C. or lower, 180° C. or lower or 170° C. or lower, and high heat resistance may be obtained therefrom.

In the present disclosure, pencil hardness of the polycarbonate may exhibit high hardness of B or HB when measured at an angle of 45 degrees with a load of 50 g in accordance with ASTM D3363.

In the present disclosure, transmittance of the polycarbonate is from 80% to 90%. The transmittance of the polycarbonate may be measured according to the ASTM evaluation method D1003. When the transmittance of the polycarbonate satisfies the above-described range, excellent optical properties are obtained.

### Method for Preparing Polycarbonate

One embodiment of the present disclosure provides a method for preparing polycarbonate, the method including polymerizing a composition including a diol compound represented by the following Chemical Formula 1; at least one compound of compounds represented by the following Chemical Formulae 2 and 3; and a carbonate precursor.

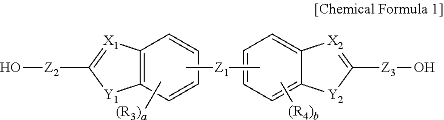

In Chemical Formula 1,
Z₁ is CR₁R₂, O, S, S—S, C═O, C═S, S—O, SO₂, (CH₂)ₙ-L₁-(CH₂)ₘ or O—(C═O),
R₁ and R₂ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic or aromatic ring unsubstituted or substituted with halogen, substituted or unsubstituted alkyl, substituted or unsubstituted aryl or substituted or unsubstituted heteroaryl,
L₁ is O or S,
n and m are each independently an integer of 1 to 3,
Z₂ and Z₃ are each independently a single bond, substituted or unsubstituted alkylene, substituted or unsubstituted cycloalkylene, substituted or unsubstituted arylene or substituted or unsubstituted heteroarylene, or a combination thereof,
X₁ and X₂ are each independently CR₁₀₀ or N,
Y₁ and Y₂ are each independently CR₁₀₁R₁₀₂, O, or S,
R₁₀₀, R₁₀₁, R₁₀₂, R₃ and R₄ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂,
a and b are each independently an integer of 0 to 3, and
R^{a} to R^{f} are each independently hydrogen, substituted or unsubstituted alkyl, substituted or unsubstituted cycloalkyl, substituted or unsubstituted aryl or substituted or unsubstituted heteroaryl,

HO-A₁-OH [Chemical Formula 2]
in Chemical Formula 2,
A₁ is substituted or unsubstituted alkylene, substituted or unsubstituted cycloalkylene or isosorbide,

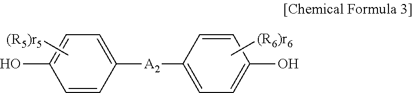

in Chemical Formula 3,
A₂ is substituted or unsubstituted alkylene, substituted or unsubstituted cycloalkylene, substituted or unsubstituted arylene, substituted or unsubstituted heteroarylene, O, S, S—O, SO₂ or C═O,
R₅ and R₆ are each independently hydrogen, substituted or unsubstituted alkyl, substituted or unsubstituted cycloalkyl, substituted or unsubstituted aryl, substituted or unsubstituted heteroaryl, alkoxy or halogen, and
r₅ and r₆ are each independently an integer of 0 to 4.

In one embodiment of the present disclosure, the composition includes the diol compound represented by Chemical Formula 1; the compound represented by Chemical Formula 3 and a carbonate precursor.

In the present disclosure, the compound represented by Chemical Formula 2 may be represented by the following chemical formula, but is not limited thereto.

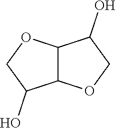

In the present disclosure, the compound represented by Chemical Formula 3 may be one or more types of compounds selected from the group consisting of bis(4-hydroxyphenyl)methane, bis(4-hydroxyphenyl)ether, bis(4-hydroxyphenyl)sulfone, bis (4-hydroxyphenyl)sulfoxide, bis(4-hydroxyphenyl)sulfide, bis(4-hydroxyphenyl)ketone, 1,1-bis(4-hydroxyphenyl)ethane, 2,2-bis(4-hydroxyphenyl)propane (bisphenol A), 2,2-bis(4-hydroxyphenyl)butane, 1,1-bis(4-hydroxyphenyl)cyclohexane (bisphenol Z), 2,2-bis(4-hydroxy-3,5-dibromophenyl)propane, 2,2-bis(4-hydroxy-3,5-dichlorophenyl)propane, 2,2-bis(4-hydroxy-3-bromophenyl)propane, 2,2-bis(4-hydroxy-3-chlorophenyl)propane, 2,2-bis(4-hydroxy-3-methylphenyl)propane, 2,2-bis(4-hydroxy-3,5-dimethylphenyl)propane and 1,1-bis(4-hydroxyphenyl)-1-phenylethane, but is not limited thereto.

Preferably, the compound represented by Chemical Formula 3 is 2,2-bis(4-hydroxyphenyl)propane (bisphenol A).

In the present disclosure, the carbonate precursor performs a role of linking the diol compound represented by Chemical Formula 1 and the compounds represented by Chemical Formula 2 and/or Chemical Formula 3, and specific examples thereof may include phosgene, triphosgene, diphosgene, bromophosgene, dimethyl carbonate, diethyl carbonate, dibutyl carbonate, dicyclohexyl carbonate, diphenyl carbonate, ditolyl carbonate, bis(chlorophenyl) carbonate, m-cresyl carbonate, dinaphthyl carbonate, bis(diphenyl) carbonate or bishaloformate, but are not limited thereto.

Preferably, the carbonate precursor is triphosgene.

As a method of polymerizing polycarbonate using a composition including, in addition to the compound represented by Chemical Formula 1, at least one compound of the compounds represented by Chemical Formulae 2 and 3 and the carbonate precursor, a polymerization process may be performed once for the composition including the three or four compounds.

In the present disclosure, the diol compound represented by Chemical Formula 1 may be used in 1% by weight or greater, 2% by weight or greater or 3% by weight or greater, and 15% by weight or less, 12% by weight or less or 10% by weight or less, with respect to 100% by weight of the composition.

In the present disclosure, the compound represented by Chemical Formula 2 may be used in 40% by weight or greater, 50% by weight or greater or 55% by weight or greater, and 80% by weight or less, 75% by weight or less or 70% by weight or less, with respect to 100% by weight of the composition.

In the present disclosure, the compound represented by Chemical Formula 3 may be used in 40% by weight or greater, 50% by weight or greater or 55% by weight or greater, and 80% by weight or less, 75% by weight or less or 70% by weight or less, with respect to 100% by weight of the composition.

In the present disclosure, the carbonate precursor may be used in 10% by weight or greater, 15% by weight or greater or 20% by weight or greater, and 50% by weight or less, 40% by weight or less or 35% by weight or less, with respect to 100% by weight of the composition.

Using the diol compound represented by Chemical Formula 1, the compound represented by Chemical Formula 2, the compound represented by Chemical Formula 3, and the carbonate precursor each in the above-described content is effective in enhancing mechanical properties of the polycarbonate.

In the present disclosure, the polymerization may be conducted using a method of interfacial polymerization or melt polymerization, but is not limited thereto.

Specifically, the polymerization temperature is preferably from 0° C. to 40° C. and the reaction time is preferably from 10 minutes to 5 hours in the interfacial polymerization. In addition, the pH is preferably maintained at 9 or higher or 11 or higher during the reaction.

In the present disclosure, the polymerization is conducted using a melt polymerization method.

A solvent that may be used in the polymerization is not particularly limited as long as it is a solvent used in polycarbonate polymerization in the art, and as one example, halogenated hydrocarbon such as methylene chloride or chlorobenzene may be used.

In addition, the polymerization is preferably conducted in the presence of an acid binder, and as the acid binder, an alkali metal hydroxide such as sodium hydroxide or potassium hydroxide, or an amine compound such as pyridine may be used.

In the present disclosure, examples of a carbonate diester compound usable as a starting raw material used in a transesterification reaction may include carbonates of diaryl compounds, carbonates of dialkyl compounds, carbonates of alkylaryl compounds or the like, however, the present disclosure is not limited thereto.

For the diol compound represented by Chemical Formula 1, at least one compound of the compounds represented by Chemical Formulae 2 and 3, and the carbonate diester, “carbonate diester/the diol compound represented by Chemical Formula 1, at least one compound of the compounds represented by Chemical Formulae 2 and 3” may have a molar ratio of 0.9 to 1.5, preferably 0.95 to 1.20, and more preferably 0.98 to 1.20.

In preparing the polycarbonate through the transesterification reaction of the present disclosure, additives such as an end-stopper, a branching agent and an antioxidant may be additionally used as necessary.

The end-stopper, the branching agent, the antioxidant and the like may be added in the form of powder, liquid, gas or the like, and these function to enhance quality of the obtained polycarbonate resin.

The reaction pressure is not particularly limited in the transesterification reaction, and may be adjusted depending on the vapor pressure of used monomers, and the reaction temperature, but usually, a pressurized state with atmospheric pressure of 1 atmosphere to 10 atmospheres is employed in the initial stage of the reaction, and in the latter stage of the reaction, the pressure is reduced to a final pressure of 0.1 mbar to 100 mbar.

In addition, the transesterification reaction may be conducted until a target molecular weight is obtained, and the reaction time is commonly from 0.2 hours to 10 hours.

The transesterification reaction is normally conducted in the absence of an inert solvent, however, as necessary, the transesterification reaction may also be conducted in the presence of an inert solvent in 1% by weight to 150% by weight of the obtained polycarbonate resin.

As the inert solvent, aromatic compounds such as diphenyl ether, halogenated diphenyl ether, benzophenone, polyphenylene ether, dichlorobenzene and methylnaphthalene; or cycloalkanes such as tricyclo(5,2,10)decane, cyclooctane and cyclodecane may be used.

In addition, the transesterification reaction may also be conducted under the inert gas atmosphere as necessary, and as the inert gas, a gas such as argon, carbon dioxide, dinitrogen monoxide or nitrogen; chlorofluoro hydrocarbon, alkane such as ethane or propane, or alkene such as ethylene or propylene, and the like may be used.

As the transesterification reaction proceeds under the condition as above, phenols, alcohols, or esters thereof corresponding to the used carbonate diester; and the inert solvent are extracted from the reactor. These extracted materials may be separated, purified and recycled. The transesterification reaction may be conducted batchwise or continuously using any apparatus.

Herein, the reaction apparatus of the transesterification reaction may be used as long as it has a common stirring function, and having a high-viscosity type stirring function is preferred since viscosity increases in the latter stage of the reaction.

In addition, a preferred type of the reactor is a vessel type or an extruder type.

In addition, the reaction pressure is preferably from 0.1 mbar to 100 mbar during pre-polymerization, and is more preferably from 1 mbar to 10 mbar. When the reaction pressure is in the rage of 0.1 mbar to 100 mbar, the composition in the transesterification reaction system does not change since carbonate diester, the starting raw material, is not removed by distillation, and it is more preferred in terms that the reaction proceeds smoothly since a monohydroxy compound that is a by-product is removed by distillation.

### Molded Article

One embodiment of the present disclosure provides a molded article including the polycarbonate.

As described above, polycarbonate including the diol compound represented by Chemical Formula 1-derived repeating unit has enhanced surface hardness properties, and has a wider field of application compared to molded articles manufactured using existing polycarbonate used in the art.

In addition, polycarbonate having target properties may be prepared by adjusting a molar ratio of the repeating unit of the diol compound represented by Chemical Formula 1 and the repeating unit of at least one compound of the compounds represented by Chemical Formulae 2 and 3.

In the present disclosure, the molded article may further include, in addition to the polycarbonate according to the present disclosure, one or more types selected from the group consisting of an antioxidant, a plasticizer, an antistatic agent, a nucleating agent, a flame retardant, a lubricant, an impact modifier, a fluorescent brightening agent, an ultraviolet absorber, a pigment and a dye as necessary.

In the present disclosure, as one example of a method for manufacturing the molded article, the method may include well mixing the polycarbonate according to the present disclosure and other additives using a mixer, extrusion molding the mixture using an extruder to prepare into a pellet, drying the pellet, and injecting the pellet using an injection molding machine.

In the present disclosure, the polymerization is preferably conducted in the presence of a molecular weight modifier in order to adjust the molecular weight of the polycarbonate in the polymerization. As the molecular weight modifier, a C1-20 alkylphenol may be used, and specific examples thereof may include p-tert-butylphenol, p-cumylphenol, decylphenol, dodecylphenol, tetradecylphenol, hexadecylphenol, octadecylphenol, eicosylphenol, docosylphenol or triacontylphenol. The molecular weight modifier may be introduced before initiation of the polymerization, during initiation of the polymerization or after initiation of the polymerization. The molecular weight modifier may be used in 0.01 parts by weight to 10 parts by weight, or preferably in 0.1 parts by weight to 6 parts by weight, with respect to 100 parts by weight of the repeating unit of the compound represented by Chemical Formula 1 and the repeating units of the compounds represented by Chemical Formula 2 and/or Chemical Formula 3, and a target molecular weight may be obtained in this range.

In the present disclosure, a reaction accelerator such as a tertiary amine compound, a quaternary ammonium compound or a quaternary phosphonium compound such as triethylamine, tetra-n-butylammonium bromide or tetra-n-butylphosphonium bromide may be additionally used in order to facilitate the polymerization reaction.

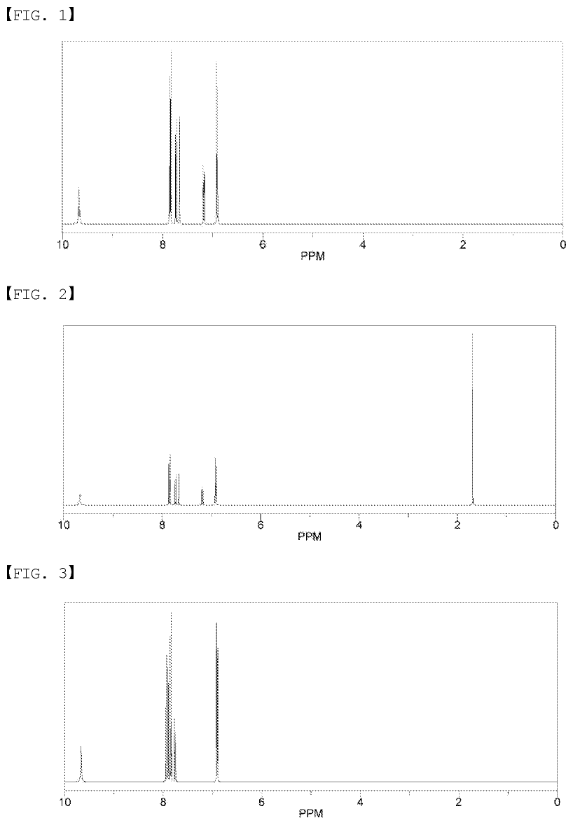

FIG. 1 to FIG. 3 are ¹H-NMR graphs of the diol compounds respectively prepared in Examples 1 to 3.

Specifically, FIG. 1 has a peak at 9.8 ppm, and shows that the diol compound includes an —OH structure.

FIG. 2 has a peak at 1.8 ppm, and shows that the diol compound includes a dimethyl group structure, and by having a peak at 9.8 ppm, it is shown that the diol compound includes an —OH structure.

FIG. 3 has a peak at 9.8 ppm, and it is shown that the diol compound includes an —OH structure.

Hereinafter, the present disclosure will be described in more detail in the following examples. However, the following examples are for illustrative purposes only, and the scope of the present disclosure is not limited to the following examples.

### EXAMPLES

### Example 1

### (1) Preparation of Compound 1 (4,4′-((perfluoropropane-2,2-diyl)bis(benzo[d]oxazole-5,2-diyl))diphenol)

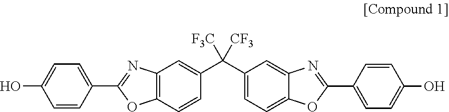 [SMILES: Oc1ccc(-c2nc3cc(C(c4ccc5oc(-c6ccc(O)cc6)nc5c4)(C(F)(F)F)C(F)(F)F)ccc3o2)cc1]

To a 2-neck flask, 4,4′-(perfluoropropane-2,2-diyl)bis(2-aminophenol) (100 g), 4-hydroxybenzoic acid (95 g) and p-toluenesulfonic acid (141 g) were introduced, and after adding xylene (1,000 ml) thereto, a dean-stark apparatus was installed. After that, the mixture was stirred for 24 hours under reflux. After the reaction was finished, the result was extracted with EA (ethyl acetate)/H₂O, EA/Sat. K₂CO₃ solution once each, and then recrystallized with ethyl acetate/toluene to obtain Compound 1.

¹H-NMR of Compound 1 is shown in FIG. 1.

### (2) Preparation of Polycarbonate

To a polymerization reactor, water (2,044 g), sodium hydroxide (NaOH) (140 g) and bisphenol A (BPA) (208.8 g) were introduced, and mixed and dissolved under the N₂ atmosphere.

Para-tert-butylphenol (PTBP) (4.6 g) and Compound 1 (23.2 g) prepared above, which were dissolved in methylene chloride (MC), were introduced thereto.

Then, triphosgene (TPG) (128 g) dissolved in methylene chloride was introduced thereto for 1 hour and reacted while maintaining the pH at 11 or higher, and after 10 minutes, triethylamine (TEA) (46 g) was introduced thereto to conduct a coupling reaction. After a total reaction time of 1 hour and 20 minutes, the pH was lowered to 4 to remove the triethylamine, and by washing the result 3 times with distilled water, the pH of the produced polymer was adjusted to neutral of 6 to 7. The polymer obtained as above was re-precipitated in a mixture solution of methanol and hexane, and the obtained result was dried at 120° C. to obtain polycarbonate.

For the obtained polycarbonate, the molecular weight was measured by gel permeation chromatography (GPC) using a PC standard, and a weight average molecular weight of 48,000 g/mol was identified.

### Example 2

### (1) Preparation of Compound 2 (4,4′-(propane-2,2-diylbis(benzo[d]oxazole-5,2-diyl))diphenol)

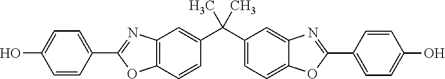 [SMILES: CC(C)(c1ccc2oc(-c3ccc(O)cc3)nc2c1)c1ccc2oc(-c3ccc(O)cc3)nc2c1]

Compound 2 was synthesized in the same manner as in Example 1 except that 4,4′-(propane-2,2-diyl)bis(2-aminophenol) (70.5 g) was used instead of 4,4′-(perfluoropropane-2,2-diyl)bis(2-aminophenol) (100 g).

¹-NMR of Compound 2 is shown in FIG. 2.

### (2) Preparation of Polycarbonate

Polycarbonate was synthesized in the same manner as in Example 1 except that Compound 2 was used instead of Compound 1.

For the obtained polycarbonate resin, the molecular weight was measured by gel permeation chromatography (GPC) using a PC standard, and a weight average molecular weight of 47,100 g/mol was identified.

### Example 3

### (1) Preparation of Compound 3 (4,4′-(sulfonylbis(benzo[d]oxazole-5,2-diyl))diphenol)

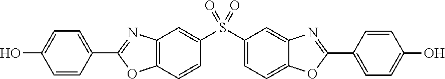 [SMILES: O=S(=O)(c1ccc2oc(-c3ccc(O)cc3)nc2c1)c1ccc2oc(-c3ccc(O)cc3)nc2c1]

Compound 3 was synthesized in the same manner as in Example 1 except that 4,4′-sulfonylbis(2-aminophenol) (76.5 g) was used instead of 4,4′-(perfluoropropane-2,2-diyl)bis(2-aminophenol) (100 g).

¹-NMR of Compound 3 is shown in FIG. 3.

### (2) Preparation of Polycarbonate

Polycarbonate was synthesized in the same manner as in Example 1 except that Compound 3 was used instead of Compound 1.

For the obtained polycarbonate resin, the molecular weight was measured by gel permeation chromatography (GPC) using a PC standard, and a weight average molecular weight of 47,700 g/mol was identified.

### Comparative Example

### Comparative Example 1

Polycarbonate and an injected specimen thereof were prepared in the same manner as in Example 1 except that Compound 1 was not used. For the obtained polycarbonate, the molecular weight was measured by gel permeation chromatography (GPC) using a PC standard, and a weight average molecular weight of 49,700 g/mol was identified.

### Comparative Example 2

Polycarbonate and an injected specimen thereof were prepared in the same manner as in Comparative Example 1 except that bisphenol C was used instead of bisphenol A. For the obtained polycarbonate, the molecular weight was measured by gel permeation chromatography (GPC) using a PC standard, and a weight average molecular weight of 48,300 g/mol was identified.

### Experimental Example: Evaluation of Polycarbonate Properties

For each of 100 parts by weight of the polycarbonate resins prepared in the examples and the comparative examples, 0.050 parts by weight of tris(2,4-di-tert-butylphenyl)phosphite, 0.010 parts by weight of octadecyl-3-(3,5-di-tert-butyl-4-hydroxyphenyl)propionate and 0.030 parts by weight of pentaerythritol tetraacrylate were added, and the result was pelletized using a vent-attached HAAKE Mini CTW and then injection molded at a cylinder temperature of 300° C. and a mold temperature of 120° C. using a HAAKE Minijet injection molding machine to prepare a specimen.

Properties of such an injected specimen or polycarbonate were measured using the following method, and the results are shown in the following Table 1.

### Measurement Method

Repeating unit: measured by ¹H-NMR using Varian 500 MHz.
Weight average molecular weight (g/mol): calibrated with PC standard and measured using Agilent 1200 series.
Izod room temperature impact strength (Kgf/m²): measured at 23° C. in accordance with ASTM D256 (⅛ inch, Notched Izod).
Glass transition temperature (Tg, ° C.): measured using DSC (TA Instrument) apparatus.
Pencil hardness: measured using pencil having strength of 3B, B, HB and H at 45 degree angle with 50 g load using pencil hardness tester (Cometech) in accordance with ASTM D3363.
Transmittance: measured in accordance with ASTM evaluation method D1003.

TABLE 1

| | | | | Compar- | Compar- |
|---|---|---|---|---|---|
| | Exam- | Exam- | Exam- | ative | ative |
| | ple 1 | ple 2 | ple 3 | Example 1 | Example 2 |
| Izod Room | 310 | 250 | 290 | 210 | 120 |
| Temperature Impact | | | | | |
| Strength (Kgf/m²) | | | | | |
| Glass Transition | 155 | 150 | 170 | 154 | 120 |
| Temperature (° C.) | | | | | |
| Transmittance (%) | 80 | 90 | 82 | 87 | 80 |
| Pencil Hardness | HB | B | B | 3B | H |

According to Table 1, Examples 1 to 3 have higher impact strength compared to Comparative Examples 1 and 2, and excellent mechanical properties are identified.

In addition, Examples 1 and 2 have a higher glass transition temperature compared to Comparative Examples 1 and 2, and the polycarbonate of the present disclosure having high heat resistance is identified.

On the other hand, Comparative Example 2 has very low impact strength and glass transition temperature despite excellent pencil hardness compared to Examples 1 to 3, and it is identified that polycarbonate properties aimed in the present disclosure are not satisfied.

1. Polycarbonate comprising:
a diol compound represented by Chemical Formula 1;
at least one compound of compounds represented by Chemical Formulae 2 and 3; and
a carbonate precursor-derived repeating unit: 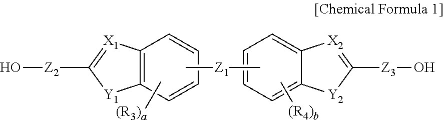
wherein, in Chemical Formula 1,
Z₁ is CR₁R₂, O, S, S—S, C═O, C═S, S—O, SO₂, (CH₂)ₙ-L₁-(CH₂)ₘ or O—(C═O);
R₁ and R₂ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic or aromatic ring unsubstituted or substituted with halogen, substituted or unsubstituted alkyl, substituted or unsubstituted aryl or substituted or unsubstituted heteroaryl;
L₁ is O or S;
n and m are each independently an integer of 1 to 3;
Z₂ and Z₃ are each independently a single bond, substituted or unsubstituted alkylene, substituted or unsubstituted cycloalkylene, substituted or unsubstituted arylene or substituted or unsubstituted heteroarylene, or a combination thereof;
X₁ and X₂ are each independently CR₁₀₀ or N;
Y₁ and Y₂ are each independently CR₁₀₁R₁₀₂, O or S;
R₁₀₀, R₁₀₁, R₁₀₂, R₃ and R₄ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂;
a and b are each independently an integer of 0 to 3; and
R^{a} to R^{f} are each independently hydrogen, substituted or unsubstituted alkyl, substituted or unsubstituted cycloalkyl, substituted or unsubstituted aryl or substituted or unsubstituted heteroaryl,
HO-A₁-OH [Chemical Formula 2]
in Chemical Formula 2,
A₁ is substituted or unsubstituted alkylene, substituted or unsubstituted cycloalkylene or isosorbide, 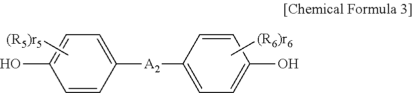
in Chemical Formula 3,
A₂ is substituted or unsubstituted alkylene, substituted or unsubstituted cycloalkylene, substituted or unsubstituted arylene, substituted or unsubstituted heteroarylene, O, S, S—O, SO₂ or C═O;
R₅ and R₆ are each independently hydrogen, substituted or unsubstituted alkyl, substituted or unsubstituted cycloalkyl, substituted or unsubstituted aryl, substituted or unsubstituted heteroaryl, alkoxy or halogen; and
r₅ and r₆ are each independently an integer of 0 to 4.

2. The polycarbonate of claim 1, wherein, in Chemical Formula 1,
Z₁ is CR₁R₂, O, S, S—S, C═O, C═S, S—O, SO₂, (CH₂)ₙ-L₁-(CH₂)ₘ or O—(C═O);
R₁ and R₂ are each independently hydrogen, substituted or unsubstituted alkyl, substituted or unsubstituted haloalkyl, OR^{a}, SR^{b}, NR^{c}R^{d}, COOR^{e}, OCOR^{f}, halogen, CN or NO₂, or R¹ and R₂ are linked to each other to form an aliphatic ring of 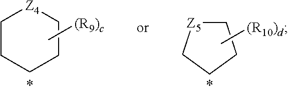
* means a part linked to Chemical Formula 1;
L₁ is O or S;
n and m are each independently an integer of 1 to 3;
Z₄ and Z₅ are each independently CR₁₀₃R₁₀₄, NR₁₀₅, O or S;
R₁₀₃ to R₁₀₅, R₉ and R₁₀ are each independently hydrogen, halogen, substituted or unsubstituted alkyl, substituted or unsubstituted aryl or substituted or unsubstituted heteroaryl; and
c is an integer of 0 to 8, d is an integer of 0 to 6, and the rest of the substituents have the same definitions as in Chemical Formula 1.

3. The polycarbonate of claim 1, wherein
Z₂ and Z₃ are each independently 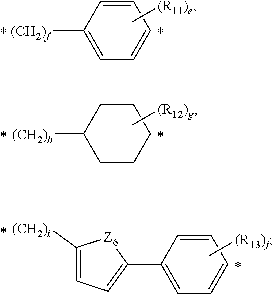
* means a part linked to Chemical Formula 1;
R₁₁ to R₁₃ are each independently hydrogen, alkoxy, substituted or unsubstituted alkyl or OH;
Z₆ is NRn, O or S;
Rn is hydrogen or a substituted or unsubstituted alkyl group;
e and j are each independently an integer of 0 to 4;
g is an integer of 0 to 10; and
f, h and i are each independently an integer of 0 to 10.

4. The polycarbonate of claim 1, wherein, when X₁ and X₂ are N, Y₁ and Y₂ are each independently O or S; and
when X₁ and X₂ are CR₁₀₀, Y₁ and Y₂ are each independently CR₁₀₁R₁₀₂ or O.

5. The polycarbonate of claim 1, wherein R₃ and R₄ are each independently hydrogen, substituted or unsubstituted alkyl, halogen, CN or NO₂.

6. The polycarbonate of claim 1, wherein the diol compound represented by Chemical Formula 1 is any one of the following compounds:

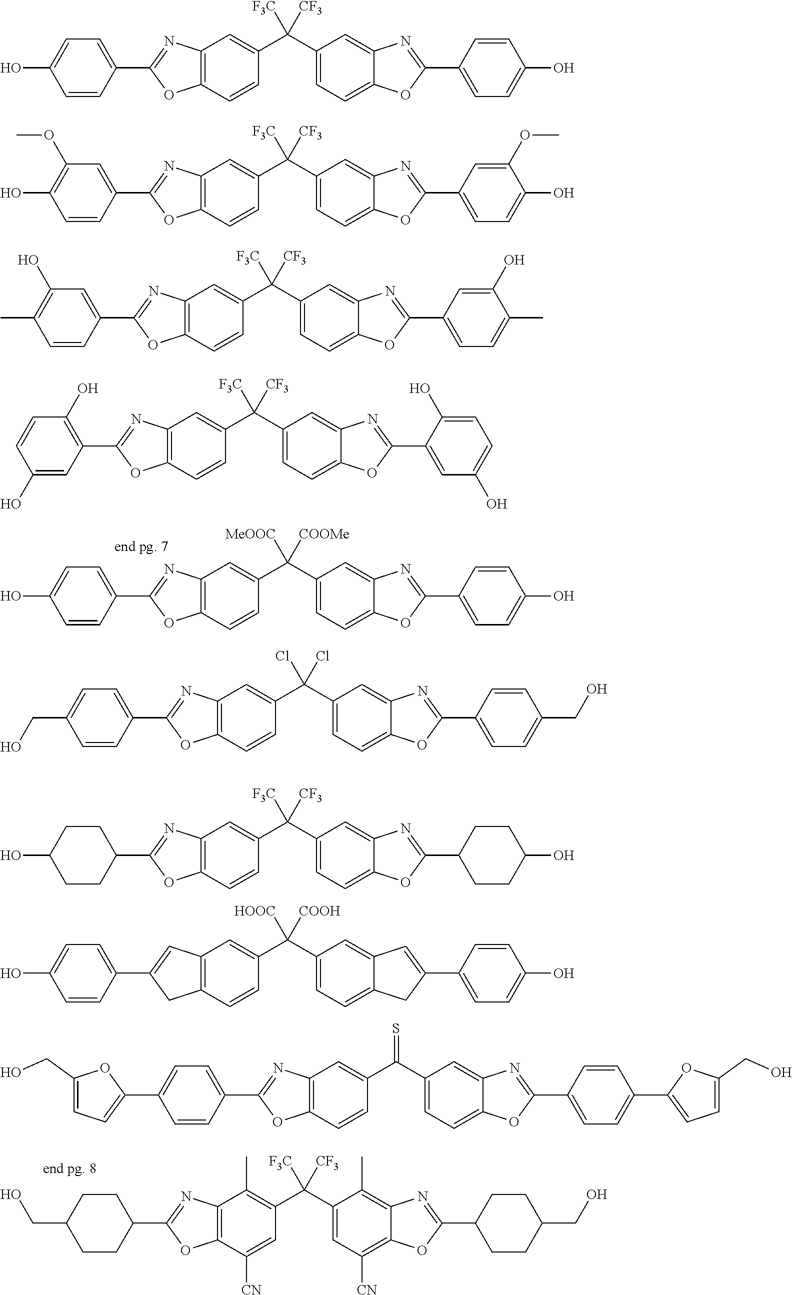

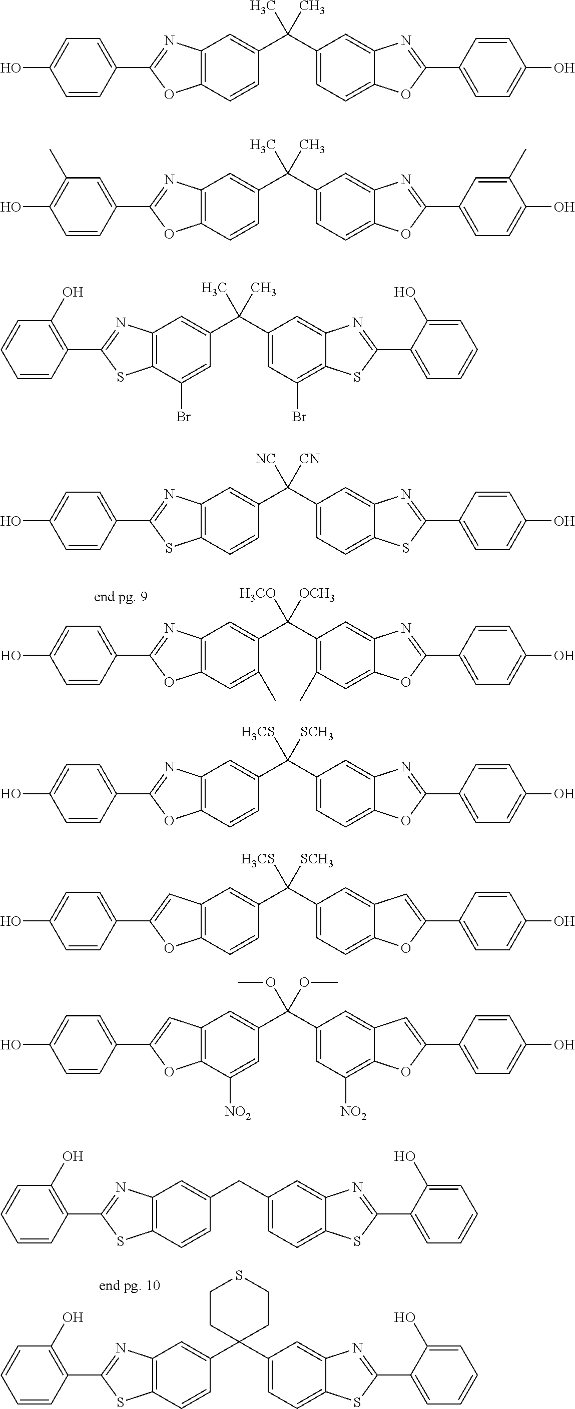

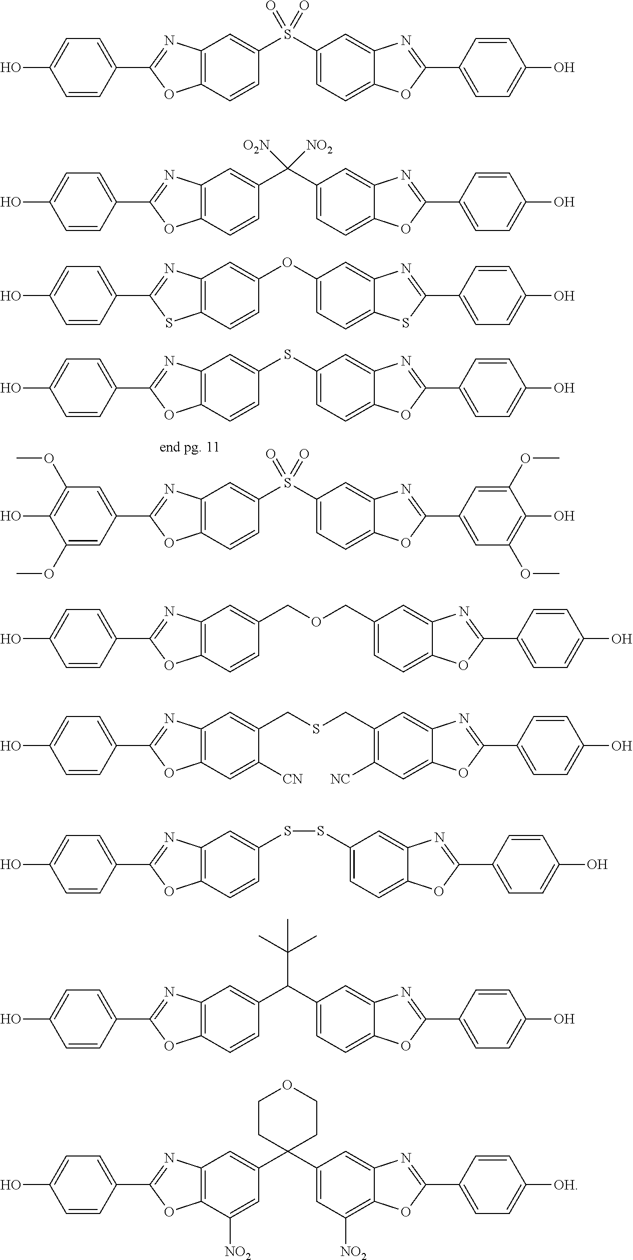

7. The polycarbonate of claim 1, wherein the diol compound represented by Chemical Formula 1; at least one compound of the compounds represented by Chemical Formulae 2 and 3; and the carbonate precursor-derived repeating unit includes a unit represented by Chemical Formula 4: 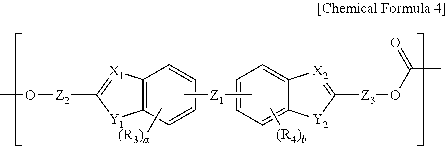
in Chemical Formula 4,
Z₁ to Z₃, R₃, R₄, X₁, X₂, Y₁, Y₂, a and b have the same definitions as in Chemical Formula 1.

8. The polycarbonate of claim 7, wherein the diol compound represented by Chemical Formula 1; at least one compound of the compounds represented by Chemical Formulae 2 and 3; and the carbonate precursor-derived repeating unit further includes a repeating unit represented by Chemical Formula 5: 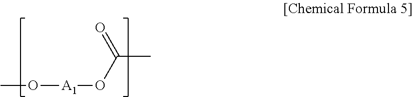
in Chemical Formula 5,
A₁ has the same definition as in Chemical Formula 2.

9. The polycarbonate of claim 7, wherein the diol compound represented by Chemical Formula 1; at least one compound of the compounds represented by Chemical Formulae 2 and 3; and the carbonate precursor-derived repeating unit further includes a repeating unit represented by Chemical Formula 6: 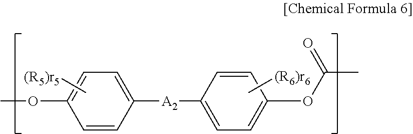
in Chemical Formula 6,
A₂, R₅, R₆, r₅ and r₆ have the same definitions as in Chemical Formula 3.

10. The polycarbonate of claim 1, wherein Izod room temperature impact strength measured at 23° C. in accordance with ASTM D256 (⅛ inch, Notched Izod) is 220 Kgf/m² or greater.

11. A method for preparing polycarbonate, the method comprising polymerizing a composition including a diol compound represented by the following Chemical Formula 1; at least one compound of compounds represented by Chemical Formulae 2 and 3; and a carbonate precursor: 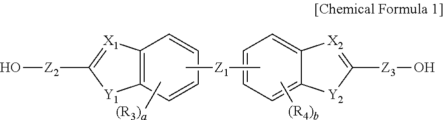
wherein, in Chemical Formula 1,
Z₁ is CR₁R₂, O, S, S—S, C═O, C═S, S—O, SO₂, (CH₂)ₙ-L₁-(CH₂)ₘ or O—(C═O);
R₁ and R₂ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂, or R₁ and R₂ are linked to each other to form an aliphatic or aromatic ring unsubstituted or substituted with halogen, substituted or unsubstituted alkyl, substituted or unsubstituted aryl or substituted or unsubstituted heteroaryl;
L₁ is O or S;
n and m are each independently an integer of 1 to 3;
Z₂ and Z₃ are each independently a single bond, substituted or unsubstituted alkylene, substituted or unsubstituted cycloalkylene, substituted or unsubstituted arylene or substituted or unsubstituted heteroarylene, or a combination thereof;
X₁ and X₂ are each independently CR₁₀₀ or N;
Y₁ and Y₂ are each independently CR₁₀₁R₁₀₂, O or S;
R₁₀₀, R₁₀₁, R₁₀₂, R₃ and R₄ are each independently hydrogen, substituted or unsubstituted alkyl, substituted or unsubstituted cycloalkyl, substituted or unsubstituted aryl, substituted or unsubstituted heteroaryl, substituted or unsubstituted haloalkyl, OR^{a}, SR^{b}, NR^{c}R^{d}, COOR^{e}, OCOR^{f}, halogen, CN or NO₂;
a and b are each independently an integer of 0 to 3; and
R^{a} to R^{f} are each independently hydrogen, substituted or unsubstituted alkyl, substituted or unsubstituted cycloalkyl, substituted or unsubstituted aryl or substituted or unsubstituted heteroaryl,
HO-A₁-OH [Chemical Formula 2]
in Chemical Formula 2,
A₁ is substituted or unsubstituted alkylene, substituted or unsubstituted cycloalkylene or isosorbide, 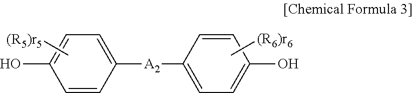
in Chemical Formula 3,
A₂ is substituted or unsubstituted alkylene, substituted or unsubstituted cycloalkylene, substituted or unsubstituted arylene, substituted or unsubstituted heteroarylene, O, S, S—O, SO₂ or C═O;
R₅ and R₆ are each independently hydrogen, substituted or unsubstituted alkyl, substituted or unsubstituted cycloalkyl, substituted or unsubstituted aryl, substituted or unsubstituted heteroaryl, alkoxy or halogen; and
r₅ and r₆ are each independently an integer of 0 to 4.

12. The method for preparing polycarbonate of claim 11, wherein the compound represented by Chemical Formula 3 is one or more types of compounds selected from the group consisting of bis(4-hydroxyphenyl)methane, bis(4-hydroxyphenyl)ether, bis(4-hydroxyphenyl)sulfone, bis(4-hydroxyphenyl)sulfoxide, bis(4-hydroxyphenyl)sulfide, bis(4-hydroxyphenyl)ketone, 1,1-bis(4-hydroxyphenyl)ethane, 2,2-bis(4-hydroxyphenyl)propane (bisphenol A), 2,2-bis(4-hydroxyphenyl)butane, 1,1-bis(4-hydroxyphenyl)cyclohexane (bisphenol Z), 2,2-bis(4-hydroxy-3,5-dibromophenyl)propane, 2,2-bis(4-hydroxy-3,5-dichlorophenyl)propane, 2,2-bis(4-hydroxy-3-bromophenyl)propane, 2,2-bis(4-hydroxy-3-chlorophenyl)propane, 2,2-bis(4-hydroxy-3-methylphenyl)propane, 2,2-bis(4-hydroxy-3,5-dimethylphenyl)propane and 1,1-bis(4-hydroxyphenyl)-1-phenylethane.

13. The method for preparing polycarbonate of claim 11, wherein the polymerization is conducted using a melt polymerization method.

14. A molded article comprising the polycarbonate of claim 1.
